(19) United States
(12) Patent Application Publication
(10) Pub. No.: US 2023/0254505 A1
(43) Pub. Date: Aug. 10, 2023

(54) AMINOGLYCOSIDE POTENTIATION FOR TREATMENT OF PULMONARY BACTERIAL INFECTION

(71) Applicant: EnBiotix, Inc., Allschwil (CH)

(72) Inventors: Diane M. JOSEPH-MCCARTHY, Cambridge, MA (US); Martina I. KOEVA, Cambridge, MA (US)

(21) Appl. No.: 18/135,907

(22) Filed: Apr. 18, 2023

Related U.S. Application Data
(63) Continuation of application No. 16/647,665, filed on Mar. 16, 2020, now Pat. No. 11,064,217.
(60) Provisional application No. 62/432,163, filed on Dec. 9, 2016.

(51) Int. Cl. H04N 19/577; H04N 19/593

(52) U.S. Cl. CPC H04N 19/577; H04N 19/593; H04N 19/124

(57) ABSTRACT

The present invention provides methods and formulations for treating or preventing bacterial infection in the lungs of a subject, including for controlling P. aeruginosa infection and/or colonization in the lungs of a patient having a chronic lung condition, such as cystic fibrosis (CF), non-cystic fibrosis bronchiectasis (non-CFBE), chronic obstructive pulmonary disorder (COPD), among others. In some embodiments, the invention provides methods and formulations for treating mycobacterial infection.

### SUMMARY OF THE INVENTION

The present invention provides methods and formulations for treating or preventing bacterial infection in the lungs of a subject, including for controlling P. aeruginosa infection and/or colonization in the lungs of a patient having a chronic lung condition, such as cystic fibrosis (CF), non-cystic fibrosis bronchiectasis (non-CFBE), chronic obstructive pulmonary disorder (COPD), non-tuberculous mycobacterial (NTM) pulmonary infection, among others.

In certain aspects, the invention provides a method for controlling bacterial infection and/or colonization in the lungs of a patient, the method comprising administering to the lungs of the patient by inhalation a formulation comprising an aminoglycoside antibiotic selected from tobramycin and amikacin, and a proton-motive force stimulating metabolite. The molar ratio of the aminoglycoside and the metabolite in the formulation is in the range of about 1:1 to about 1:15. Tobramycin and amikacin are aminoglycoside antibiotics prescribed for the treatment and control of P. aeruginosa infection of the lungs, including for cystic fibrosis patients, among other conditions. Over time, treatment with tobramycin or amikacin can induce a persister bacterial phenotype, where bacterial cells (including Pseudomonas and Mycobacterium) enter a metabolically dormant state in which bacterial cells do not take up aminoglycoside antibiotics. The proton-motive force stimulating metabolites induce bacterial persisters to increase their uptake of aminoglycoside antibiotics. Exemplary proton motive force stimulating metabolites include one or a combination of fumarate, pyruvate, methylpyruvate, ethylpyruvate, succinate, glucose, and propionate. In some embodiments, the metabolite is fumarate, optionally in combination with succinate.

In accordance with embodiments of the invention, an aminoglycoside-potentiating amount of metabolite can be delivered to sites of bacterial infection/colonization in the lung through inhalation of metabolite into the lung as a co-formulation with aminoglycoside. In accordance with embodiments, substantial metabolite reaches local sites of infection and penetrates mucosal biofilms, and is available in the lung epithelial lining fluid to potentiate aminoglycoside action, as well as in some embodiments, provide a cytoprotective effect on the airway cells.

In some embodiments, the formulation is an aqueous solution delivered using a nebulizer, for example, containing tobramycin at from about 100 to about 400 mg per unit dose, or amikacin at from about 200 to about 500 mg per unit dose. The unit dose formulation may also comprise from 100 mg to about 500 mg per dose of the proton motive force stimulating metabolite, such as, 181 to 727 mM fumarate (e.g., in a 5 ml aqueous solution).

In still other embodiments, the formulation is a dry powder for inhalation. In such embodiments, the unit dose formulation contains tobramycin at from about 75 mg to about 150 mg per dose, or amikacin at about 100 to about 200 mg per dose. The dry powder formulation may further comprise the proton-motive force stimulating metabolite at from about 100 mg to about 500 mg per dose.

In some embodiments, the formulation is delivered to a patient having a chronic lung disease, such as, for example, cystic fibrosis, bronchiectasis, non-tuberculous mycobacterial pulmonary infection, or chronic obstructive pulmonary disorder (COPD). In some embodiments, the method and formulation described herein is used for treating an acute exasperation involving Pseudomonas or other bacterial infection. In some embodiments, the patient has a chronic Pseudomonas infection with a mucoid phenotype.

While the formulation may generally be administered from one to three times daily (e.g., 2 times daily), in some embodiments, the invention allows for less aggressive aminoglycoside therapy, such as administration once daily. The formulation may be delivered in a regimen in which drug is administered for 28 consecutive days, which can be followed by about 28 consecutive days off; which can then be followed by another cycle. However, in some embodiments the invention provides for less aggressive therapy, allowing for the formulation to be delivered for from 7 to 21 consecutive days, followed by an off cycle. In some embodiments, the off cycle is at least about 28 days, that is, administration of the formulation is resumed after about 28 days or more. In some embodiments, a cycle of drug administration results in eradication of Pseudomonas infection, thereby sharply reducing the frequency and aggressiveness of therapy needed to combat chronic and/or recurring bacterial infection.

In some embodiments, the patient is also undergoing treatment with a second antibiotic, which in some embodiments is an antibiotic that antagonizes the effect of the aminoglycoside, such as azithromycin.

In other aspects, the invention provides unit dose formulations, and kits comprising the same, for use in the methods described herein.

Other aspects and embodiments of the invention will be apparent from the following detailed disclosure.

### DESCRIPTION OF THE FIGURES

FIG. 1 shows the potentiation effect of fumarate (Met1) at increasing concentrations of tobramycin, using Pseudomonas strains PAO1 and PA14.

FIG. 2 shows the potentiation effect of fumarate in both tobramycin sensitive mucoid and non-mucoid Pseudomonas CF clinical isolate strains. 15 mM fumarate shows over 5 orders of magnitude potentiation.

FIG. 3 shows that around 94% of tobramycin-sensitive CF clinical isolates show a significant potentiation effect with 15 mM fumarate.

FIG. 4 shows the potentiation effect with tobramycin-sensitive COPD clinical isolates.

FIG. 5 shows that 15 mM fumarate exhibits a cytoprotective effect on human airway epithelial cells infected with Pseudomonas and treated with tobramycin.

FIG. 6 shows that fumarate provides over 3 logs potentiation of tobramycin in the presence of Azithromycin.

FIG. 7 shows potentiation in two different biofilm assays, demonstrating that 15 mM fumarate potentiates tobramycin action in the presence of a naïve bacterial biofilm.

FIG. 8 shows eradication of P. aeruginosa in colony biofilms from CF clinical isolates.

FIG. 9 shows that some tolerance to tobramycin is observed in artificial sputum media with amino acids, and this tolerance is potentiated.

FIG. 10 shows that 3 logs of potentiation are observed in the presence of CF patient sputum.

### DETAILED DESCRIPTION OF THE INVENTION

The present invention provides methods and formulations for treating or preventing bacterial infection in the lungs of a subject, including for controlling P. aeruginosa infection and/or colonization in the lungs of a patient having a chronic lung condition, such as cystic fibrosis (CF), non-cystic fibrosis bronchiectasis (non-CFBE), chronic obstructive pulmonary disorder (COPD), among others. In some embodiments, the invention provides methods and formulations for treating mycobacterial infection.

In certain aspects, the invention provides a method for controlling bacterial infection and/or colonization in the lungs of a patient, the method comprising administering to the lungs of the patient by inhalation a formulation comprising an aminoglycoside antibiotic selected from tobramycin and amikacin, and a proton-motive force stimulating metabolite. The molar ratio of the aminoglycoside and the metabolite in the formulation are in the range of about 1:1 to about 1:15, or in some embodiments, about 1:2 to about 1:15, or about 1:5 to about 1:15. In some embodiments, the molar ratio of the aminoglycoside and the metabolite in the formulation are in the range of about 1:1 to about 1:12, or about 1:2 to about 1:10, or in the range of about 1:2 to about 1:8, or about 1:2 to about 1:5.

Tobramycin and amikacin are aminoglycoside antibiotics prescribed for, among other things, the treatment and control of P. aeruginosa infection of the lungs, including for cystic fibrosis patients. Approximately 85% of CF patients have chronic, recurrent P. aeruginosa infection, which significantly contributes to lung function decline and mortality. Tobramycin and amikacin can be delivered intravenously or delivered locally to the lungs. Local delivery can employ liquid aerosols delivered by nebulizer or alternatively by inhalation of drug in powder form. When delivered locally to control chronic infection, tobramycin is often administered in repeated cycles of 28 days on the drug (e.g., twice daily administration) followed by 28 days off drug to limit the local and systemic toxicity of tobramycin.

Treatment with tobramycin or amikacin (among other stressors) can induce a persister bacterial phenotype, where bacterial cells (including Pseudomonas) enter a metabolically dormant state in which bacterial cells do not take up aminoglycoside antibiotics. Thus, the aminoglycoside helps control, but does not eradicate chronic P. aeruginosa infection. In fact, the clinical impact of inhaled aminoglycosides is diminishing due to this induced bacterial tolerance.

As disclosed in US 2015/0366889, which is hereby incorporated by reference in its entirety, certain metabolites can stimulate bacterial persisters to increase their uptake of aminoglycoside antibiotics by inducing bacterial proton-motive force, even in the absence of bacterial growth. For example, relatively high local concentrations of glucose, mannitol, fructose, and pyruvate were shown to potentiate the action of gentamicin action on E. coli persisters. Further, US 2016/0199328 (which is hereby incorporated by reference in its entirety) shows that certain intermediate metabolites, such as fumarate and others, can potentiate aminoglycoside action in P. aeruginosa. For example, potentiation was observed at low levels of tobramycin (40 μg/mL or about 85 μM), identified as a typical peak serum concentration of tobramycin achieved by i.v. administration, with substantially higher levels of metabolite (˜60 mM carbon) to potentiate aminoglycoside action in vitro.

In accordance with embodiments of the invention, an aminoglycoside-potentiating amount of metabolite can be delivered to anatomical sites of bacterial infection/colonization through inhalation of metabolite into the lung as a co-formulation with aminoglycoside. In the various embodiments, substantial metabolite reaches local sites of infection and penetrates mucosal biofilms and is available in the lung epithelial lining fluid to potentiate aminoglycoside action, as well as in some embodiments, provide a cytoprotective effect on the airway cells. The formulations and methods described herein in some embodiments protect airway cells from toxicity or inflammation induced by the aminoglycoside or other agent, and can provide improvements in overall therapy and lung function.

In some embodiments, the formulation is an aqueous solution delivered using a nebulizer. In some embodiments, the formulation is provided at a unit volume in the range of about 2 ml to 10 ml, and in some embodiments between about 2 ml and about 7 ml. In some embodiments, the unit dose formulation for delivery by nebulizer is about 5 ml, and comprises tobramycin. In some embodiments, the unit dose formulation for delivery by nebulizer is about 2 ml, and comprises amikacin.

Various types of nebulizers are known, and can influence the amount of aminoglycoside and/or metabolite that reaches sites of infection or colonization. As used herein, the term “nebulizer” refers to a drug delivery device to administer medication in the form of a mist inhaled into the lungs. Nebulizers use oxygen, compressed air, or ultrasonic power to break up solutions and suspensions into small aerosol droplets that can be directly inhaled from the mouthpiece of the device. The lung deposition characteristics and efficacy of an aerosol depend largely on the particle or droplet size; for example, the smaller the particle the greater its chance of peripheral penetration and retention. Particles smaller than about 5 μm in diameter deposit frequently in the lower airways, and therefore are desirable for pharmaceutical aerosols.

In some embodiments, the nebulizer is a Jet nebulizer. Jet nebulizers are connected by tubing to a compressor, which causes compressed air or oxygen to flow at high velocity through a liquid medicine to turn it into an aerosol, which is then inhaled by the patient. In some embodiments, the nebulizer is an ultrasonic wave nebulizer. An ultrasonic wave nebulizer uses an electronic oscillator to generate a high frequency ultrasonic wave, which causes the mechanical vibration of a piezoelectric element. This vibrating element is in contact with a liquid reservoir and its high frequency vibration is sufficient to produce a vapor mist.

In some embodiments, the formulation is an aqueous solution, delivered with the use of a nebulizer, and which contains tobramycin at from about 100 to about 400 mg per unit dose. In some embodiments, the formulation contains about 300 mg of tobramycin per dose, which is equal to about 128 mM in a 5 ml solution. In some embodiments, the invention allows for tobramycin to be delivered at substantially lower unit doses than 300 mg, while having the same or greater efficacy. For example, in some embodiments, the unit dose of tobramycin is from about 115 to about 250 mg per dose, or about 150 to about 250 mg per dose, or about 175 to about 250 mg per dose. Unit doses can be provided in individual ampules.

In some embodiments, the formulation is an aqueous solution, delivered with the use of a nebulizer, and which contains amikacin at from about 200 to about 500 mg per unit dose. In some embodiments, the formulation contains about 400 mg of amikacin per unit dose. In some embodiments, the invention allows for amikacin to be delivered at substantially lower unit doses than 400 mg, while having the same or greater efficacy. In some embodiments, the formulation contains amikacin at from about 200 to about 350 mg per unit dose, or about 250 to about 350 mg per unit dose. Unit doses can be provided in individual ampules.

Generally, when delivered at an effective amount, the metabolite induces proton-motive force in Pseudomonas aeruginosa clinical isolates, to thereby drive aminoglycoside uptake. In some embodiments, the metabolite comprises an intermediate metabolite, such as, for example, a compound or a variant thereof associated with the tricarboxylic acid cycle (TCA), the β-oxidative pathway, the amino acid catabolic pathway, the urea cycle, and pathways of lipid catabolism. Various other intermediate metabolites are disclosed in US 2016/0199328, which is hereby incorporated by reference in its entirety. Exemplary metabolites include acetate, citrate, isocitrate, α-ketoglutarate, succinate, fumarate, malate and oxaloacetate. In these or other embodiments, the metabolite comprises a sugar, or a product that enters glycolysis or is a product of glycolysis, such as mannitol, pyruvate (or methyl or ethyl pyruvate), glucose, and/or fructose. Various other metabolites are disclosed in US 2015/0366889, which is hereby incorporated by reference in its entirety.

In some embodiments, the metabolite comprises one or a combination of fumarate, pyruvate, methylpyruvate, ethylpyruvate, succinate, glucose, and propionate. In some embodiments, the metabolite is fumarate, optionally in combination with succinate.

The bioavailability of tobramycin in the lung of cystic fibrosis patients upon local delivery has been the subject of investigation. For example, sputum samples expectorated at 10 minutes after delivery of 300 mg of tobramycin by nebulizer showed a Mean of 1,237 μg/g of sputum. Geller DE., et al., Pharmacokinetics and Bioavailability of Aerosolized Tobramycin in Cystic Fibrosis, Chest 122(1) (2002). In contrast to expectorated sputum, sputum induction by inhalation of hypertonic saline samples respiratory secretions from more distal conducting airways, which are often sites of infection in CF. Using this sampling process, tobramycin concentrations in the lung epithelial fluid were estimated to be in the range of 128 μg/g, after 300 mg of tobramycin was delivered by nebulizer. Ruddy J, et al., Sputum Tobramycin Concentrations in Cystic Fibrosis Patients with Repeated Administration of Inhaled Tobramycin, J. Aerosol Med. And Pu/mon. Drug Del. 26(2): 69-75 (2013).

In various embodiments, the methods and formulations provide for delivery of the aminoglycoside and an effective amount of proton-motive force stimulating metabolite to distal conducting airways, including in patients with chronic Pseudomonas infection, in which these distal conducting airways are likely to harbor persistent infection.

In various embodiments, the nebulizer formulation contains from about 100 mg to about 500 mg per unit dose of proton motive force stimulating metabolite. In some embodiments, the formulation contains from about 100 to 450 mg per dose, or about 100 to about 400 mg per dose, or about 100 to about 350 mg per dose, or about 100 to about 300 mg per dose, or about 100 to about 250 mg per dose, or about 100 to about 200 mg per dose. In some embodiments, the metabolite and aminoglycoside are administered in a 2 to 7 ml (e.g., 5 ml) solution by nebulizer. The metabolite delivered by nebulizer penetrates to areas of infection and/or colonization in sufficient levels to potentiate aminoglycoside action. Exemplary metabolites include one or a combination of fumarate, pyruvate, methylpyruvate, ethylpyruvate, succinate, glucose, and propionate. In some embodiments, the metabolite is fumarate optionally in combination with succinate. For example, the unit dose formulation may comprise from 115 to 300 mg of tobramycin (or from 200 to 300 mg of tobramycin), and from 181 to 727 mM fumarate (or 300 to 600 mM fumarate) in a 5 ml aqueous solution.

In still other embodiments, the formulation is a dry powder for inhalation. In such embodiments, the unit dose formulation contains tobramycin from about 75 mg to about 150 mg per dose, or amikacin at about 100 to about 200 mg per dose. The powder unit dose formulation may take the form of subdoses, for example, where 2, 3, 4, 5 or more subdoses (e.g., capsules) are administered as a single dose using an inhaler device. As with embodiments delivered by nebulizer, the metabolite can be as described above, including one or a combination of fumarate, pyruvate, methylpyruvate, ethylpyruvate, succinate, glucose, and propionate, and may include from about 100 mg to about 500 mg per dose of the proton motive force stimulating metabolite. In some embodiments, the formulation contains from about 100 to 450 mg per unit dose, or about 100 to about 400 mg per unit dose, or about 100 to about 350 mg per unit dose, or about 100 to about 300 mg per unit dose, or about 100 to about 250 mg per unit dose, or about 100 to about 200 mg per unit dose of the proton motive force stimulating metabolite. The metabolite may be fumarate optionally in combination with succinate.

An exemplary inhaler device suitable for delivery of dry powder formulations is TOBI PODHALER (Novartis). For example, a capsule containing a single sub dose is inserted into the capsule chamber of the device, a mouthpiece screwed over the top, the capsule is then pierced and the powder contents inhaled (generally with two breaths). The remaining subdoses are then delivered to constitute a single delivery.

In some embodiments, the patient has a chronic Pseudomonas infection with a mucoid phenotype. During the years following initial colonization, Pseudomonas strains mutate into mucoid variants. This conversion results in a significant increase in morbidity and mortality, and a decline in lung function. The mucoid matrix, characterized by production of alginate, allows the formation of protected biofilm microcolonies. Biofilms are organized communities of bacterial cells and enclosed in an extracellular polysaccharide matrix. This matrix forms a slippery, solid coat around the bacterial community and protects the bacterial community from the environment, including host immune responses and mucociliary clearance. Further, antibiotics can lose effectiveness against bacteria within biofilms, due to, among other things, the failure of antibiotics to fully diffuse through the biofilm and the higher percentage of persisters present in biofilms.

In some embodiments, the formulation is delivered to a patient having a chronic lung disease, such as, for example, cystic fibrosis, bronchiectasis, non-tuberculous mycobacterial pulmonary infection, or chronic obstructive pulmonary disorder (COPD). In some embodiments, the method and formulation described herein is used for treating an acute exasperation involving Pseudomonas or other bacterial infection of the lung.

Cystic fibrosis (CF) is a genetic disorder that affects mostly the lungs, and involves frequent bacterial infections. Approximately 85% of CF patients have chronic, recurrent P. aeruginosa infection, which significantly contributes to lung function decline and mortality. Long-term issues include difficulty breathing and coughing up mucus as a result of these frequent lung infections. CF is caused by the presence of mutations in both copies of the gene for the cystic fibrosis transmembrane conductance regulator (CFTR) protein, which is involved in production of sweat, digestive fluids, and mucus. When CFTR is not functional, secretions which are usually thin instead become thick. Lung problems are responsible for death in 80% of people with cystic fibrosis.

Bronchiectasis is a disease in which there is permanent enlargement of parts of the airways of the lung. Symptoms typically include a chronic cough productive of mucus. Other symptoms include shortness of breath, coughing up blood, and chest pain. As with CF, these patients suffer frequent lung infections. Bronchiectasis may result from a number of infective and acquired causes, including pneumonia, tuberculosis, immune system problems, and cystic fibrosis. The mechanism of disease is breakdown of the airways due to an excessive inflammatory response. Involved bronchi become enlarged and thus less able to clear secretions. These secretions increase the amount of bacteria in the lungs, and result in airway blockage and further breakdown of the airways. It is classified as an obstructive lung disease, along with chronic obstructive pulmonary disease and asthma. In some embodiments, the patient has non-cystic fibrosis bronchiectasis.

Chronic obstructive pulmonary disorder (COPD) is a type of obstructive lung disease characterized by long-term poor airflow. The main symptoms include shortness of breath and cough with sputum production. COPD typically worsens over time. In contrast to asthma, the airflow reduction does not improve much with the use of a bronchodilator. An acute exacerbation of COPD, which can involve bacterial infection, often involve increased shortness of breath, increased sputum production, a change in the color of the sputum from clear to green or yellow, and/or an increase in cough.

In some embodiments, the patient has a non-tuberculous mycobacterial pulmonary infection. Nontuberculous mycobacteria (NTM), also known as environmental mycobacteria, atypical mycobacteria and mycobacteria other than tuberculosis (MOTT), are mycobacteria which do not cause tuberculosis or leprosy. NTM cause pulmonary diseases that resemble tuberculosis.

While the formulation may generally be administered from one to three times daily (e.g., 2 times daily), in some embodiments, the invention allows for less aggressive aminoglycoside therapy, such as administration once daily. The formulation may be delivered in a regimen in which drug is administered for 28 consecutive days, followed by about 28 consecutive days off; which can then be followed by another cycle. However, in some embodiments the invention provides for less aggressive therapy, allowing for the formulation to be delivered for from 7 to 21 consecutive days, followed by an off cycle. For example, the formulation may be delivered for about 7 consecutive days, about 14 consecutive days, or for about 21 consecutive days. In some embodiments, the off cycle is at least about 28 days, that is, administration of the formulation is resumed after about 28 days or more. However, in some embodiments, administration of the formulation is resumed after about 6 weeks, after about 8 weeks, after about 10 weeks, after about 12 weeks, or after about 2 months, after about 3 months, after about 4 months, after about 5 months, or after about 6 months. In some embodiments, a cycle of drug administration results in eradication of Pseudomonas or other bacterial infection, thereby sharply reducing the frequency and aggressiveness of therapy needed to combat chronic and/or recurring bacterial infection.

In some embodiments, the formulation is delivered to treat an acute exasperation of bacterial infection (e.g., Pseudomonas aeruginosa infection). In some embodiments, one or two unit doses are delivered daily for about 7, about 14, about 21, or about 28 days.

In some embodiments, the patient is also undergoing treatment with a second antibiotic, which in some embodiments is an antibiotic that antagonizes the effect of the aminoglycoside. In some embodiments, the second aminoglycoside is a beta lactam antibiotic or a macrolide antibiotic, such as azithromycin. In some embodiments, the second antibiotic is administered systemically, such as orally or by i.v. While certain antibiotics such as azithromycin are considered to antagonize aminoglycoside action, the potentiation effect remains strong with the combination treatment. Nick J A, Azithromycin may antagonize inhaled tobramycin when targeting Pseudomonas aeruginosa in cystic fibrosis, Ann Am Thorac Soc. 11(3):342-50 (2014).

In another aspect, the invention provides unit dose formulations, and kits comprising the same, for use in the methods described herein. In some embodiments, the invention provides a unit dose formulation for delivery by nebulizer, the formulation comprising in an aqueous solution from 100 to 400 mg of tobramycin (e.g., about 300 mg), or from 300 to 600 mg of amikacin (e.g., about 500 mg); and from about 100 mg to about 500 mg of one or a combination of metabolites effective to induce proton motive force in bacterial persisters, such as one or more metabolites selected from fumarate, pyruvate, methylpyruvate, ethylpyruvate, succinate, glucose, and propionate. The formulation may be packaged in unit dose ampules having a volume of from 2 to 10 ml, such as in unit dose ampules of from about 2 to about 5 ml. In some embodiments, the molar ratio of aminoglycoside to metabolite is from 1:1 to 1:15. In some embodiments, the molar ratio of the aminoglycoside to metabolite is about 1:2 to about 1:15, or about 1:5 to about 1:15, or about 1:1 to about 1:12, or about 1:2 to about 1:10, or in the range of about 1:2 to about 1:8, or about 1:2 to about 1:5.

In some embodiments, the formulation comprises from about 115 mg to about 300 mg of tobramycin (or about 200 to 300 mg tobramycin), and from about 105 mg to about 425 mg of fumarate (or from about 300 mg to about 425 mg of fumarate) in a 3 to 7 ml aqueous solution. For example, the unit dose formulation may be a 5 ml aqueous solution comprising from 50 to 128 mM tobramycin and from 181 to 727 mM fumarate.

In some embodiments, the unit dose formulation is for delivery by powder aerosol, the formulation comprising as a fine powder containing from 75 to 150 mg of tobramycin, or from 100 to 200 mg of amikacin; and from about 50 mg to about 250 mg of one or a combination of metabolites effective to induce proton motive force in bacterial persisters, such as one or more selected from fumarate, pyruvate, methylpyruvate, ethylpyruvate, succinate, glucose, and propionate. In some embodiments, the unit dose may take the form of a plurality (e.g., 2, 3, 4, 5) of subdoses (e.g., capsules) to be administered as a single dose.

In various embodiments of the powder formulation, the molar ratio of aminoglycoside to metabolite is from 1:1 to 1:15, or about 1:2 to about 1:15, or about 1:5 to about 1:15, or about 1:1 to about 1:12, or about 1:2 to about 1:10, or in the range of about 1:2 to about 1:8, or about 1:2 to about 1:5.

Kits in accordance with the invention comprise no more than 28 unit doses, and in some embodiments, contain about 21, about 14, or about 7 unit doses. Thus, one cycle of drug treatment contains 7, 14, 21, or 28 unit doses. Unit doses can be in the form of ampules comprising aqueous solution for delivery by a nebulizer, or in the form of capsules comprising dry powder. In some embodiments, capsules are provided in from 2 to 5 capsule subdoses, which together constitute a single dose.

Other aspects and embodiments will be apparent from the following non-limiting examples.

### EXAMPLES

The following examples use an in vitro potentiation assay, termed planktonic stationary phase (PSP) time kill assay. In this assay, bacterial cultures in planktonic stationary phase are used to select for bacterial persisters. Experiments are conducted using the time-kill method (CLSI M26), to evaluate bacterial strains, varying the concentration of aminoglycoside and potentiator. Cultures of persisters are generated by growing the bacteria in lysogeny broth (LB) medium for 16 hours. After 4 hr of exposure to the aminoglycoside plus potentiator combination in M9 minimal medium, cells are enumerated on LB plates.

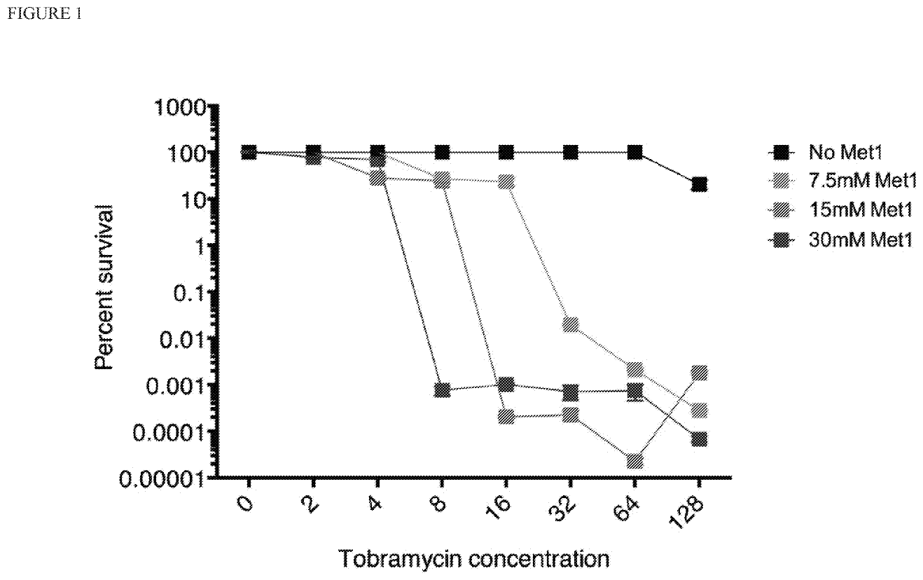

As shown in FIG. 1, 15 mM fumarate (Met1) showed over 5 logs of potentiation of tobramycin sensitivity in the persister assay with in the PAO1 laboratory strains of Pseudomonas aeruginosa. Maximum bactericidal activity was observed at 16 μg/ml tobramycin. Tobramycin has been measured at about 50 to 90 μg/ml of tobramycin in the lung epithelial lining after delivery of a 300 mg dose of tobramycin to cystic fibrosis patients by nebulizer. Ruddy J. et al., Sputum Tobramycin Concentrations in Cystic Fibrosis Patients with Repeated Administration of Inhaled Tobramycin, J. of Aerosol Med. and Pulmon. Drug Del. 26(2):69-75 (2013).

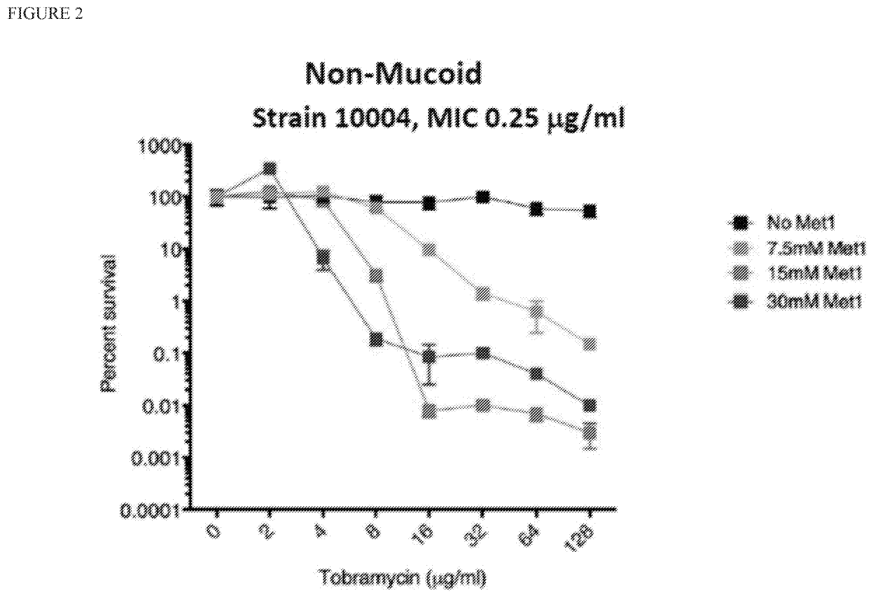

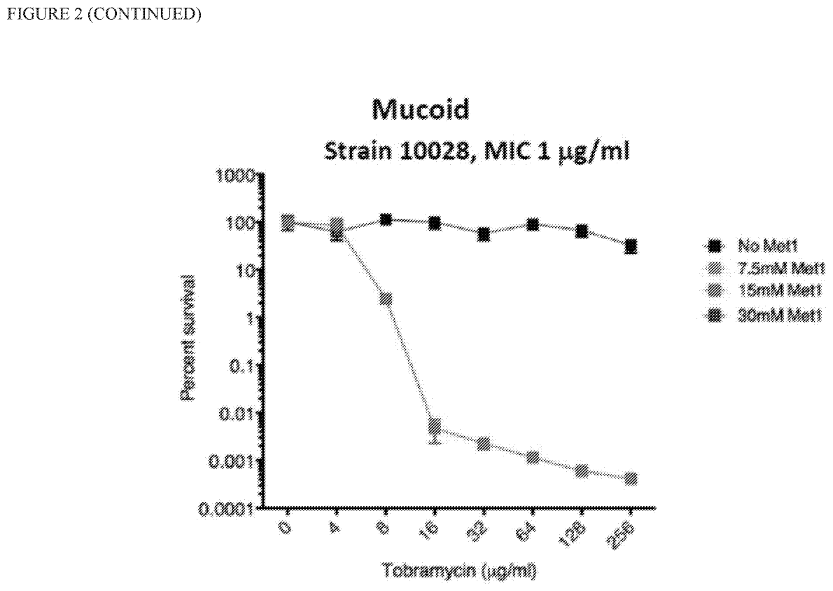

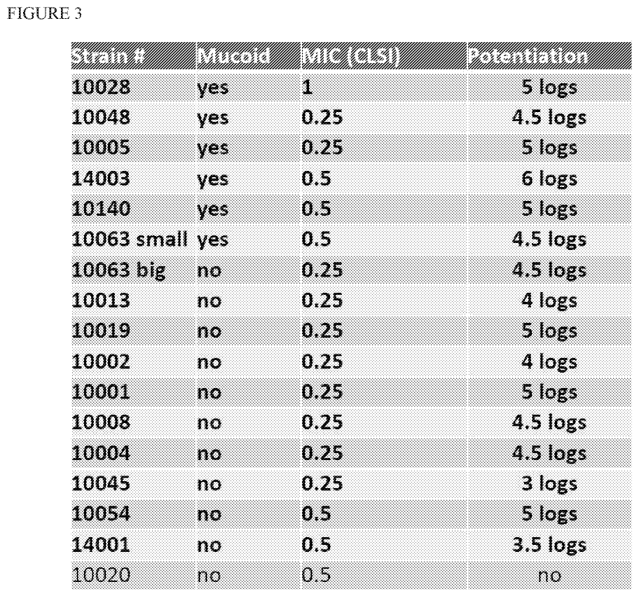

The potentiation assay was conducted with tobramycin-sensitive non-mucoid (Strain 10004) and mucoid (Strain 10028) CF clinical isolates. As shown in FIG. 2, 15 mM fumarate is effective to potentiate the action of tobramycin in both mucoid and non-mucoid strains, showing around 5 orders of magnitude potentiation. When tested over a panel of 17 CF clinical isolates (6 mucoid strains, 11 non-mucoid strains), substantial potentiation (several logs) was seen with 16 of 17 strains (94%).

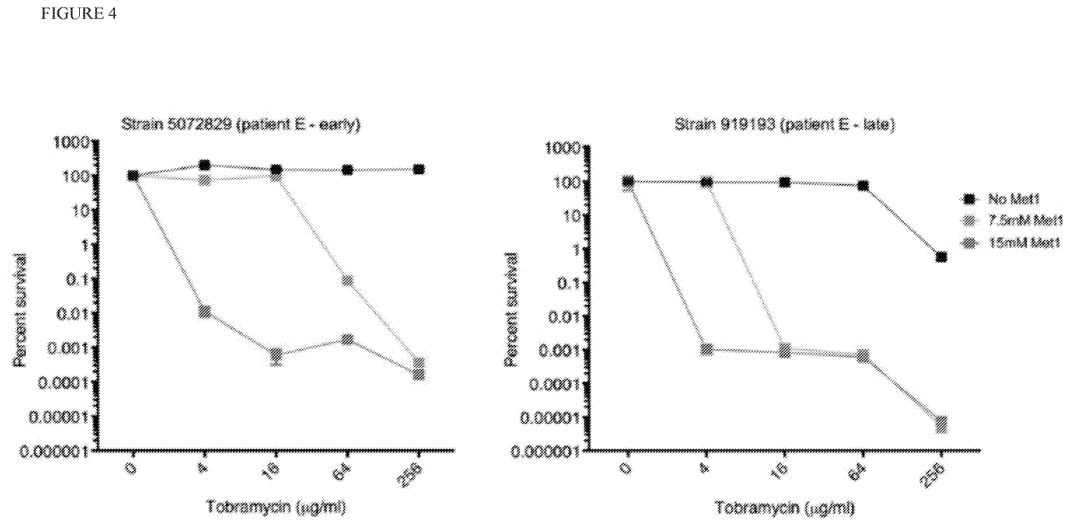

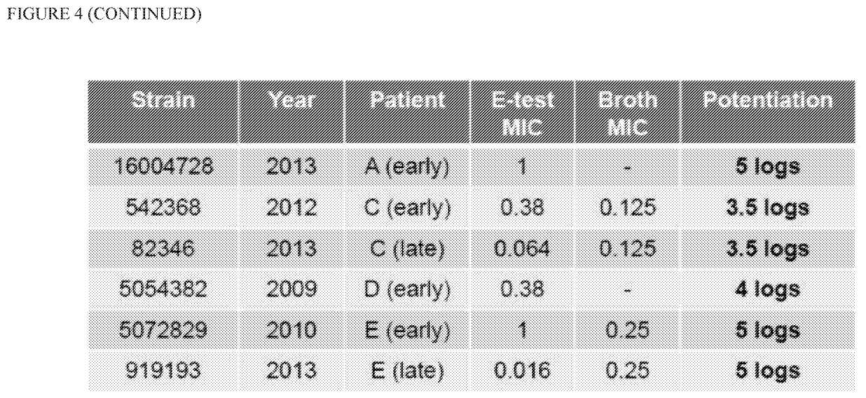

The potentiation assay was used to determine whether COPD clinical isolates (from both early or late stages) would differ in the level of potentiation. As shown in FIG. 4, 15 mM fumarate was effective to potentiate tobramycin action against all COPD strains tested.

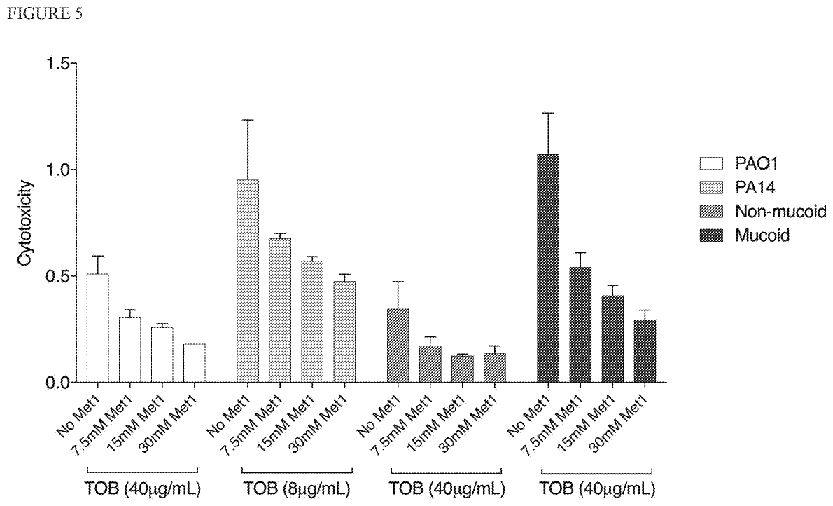

The impact of fumarate on Human Airway Epithelial cells was tested using an LDH assay. See Moreau-Marquis S, Tobramycin and FDA-Approved Iron Chelators Eliminate Pseudomonas aeruginosa Biofilms on Cystic Fibrosis Cells, Am. J. Respir. Cell Mol. Biol. Vol. 41, 305-313 (2009). LDH levels are measured in the medium (Apical and Basolateral fluids) and inside cells. Cytotoxicity can be expressed as: [LDH_{AP+BL}/(LDH_{AP+BL}+LDH_{cells})]×100. Surprisingly, 15 mM fumarate actually decreased the level of cytotoxicity observed with tobramycin, in fact a dose response was observed in cytoprotectivity with increasing fumarate concentrations. This potential of fumarate (above 7.5 mM concentration) to decrease airway inflammation provides an unexpected benefit of fumarate in combination with local pulmonary delivery of tobramycin, further improving the therapeutic window. Results are shown in FIG. 5. Results are normalized to PAO1 untreated for 100% cytotoxicity. Significance (*) is between strain plus tobramycin and strain plus tobramycin with potentiator.

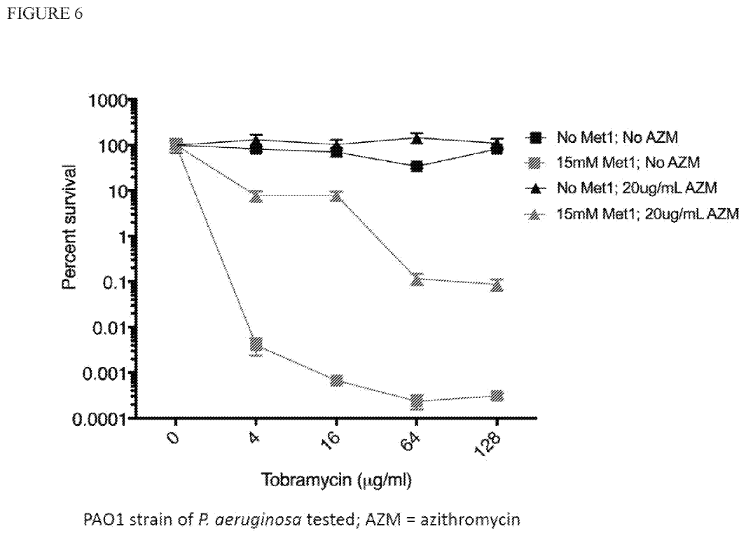

The potentiation effect was tested in the presence of both tobramycin and azithromycin. While azithromycin is considered to antagonize the action of tobramycin, as shown in FIG. 6, 15 mM fumarate provides over 3 logs potentiation of tobramycin in the presence of azithromycin. Results are for PAO1 strain.

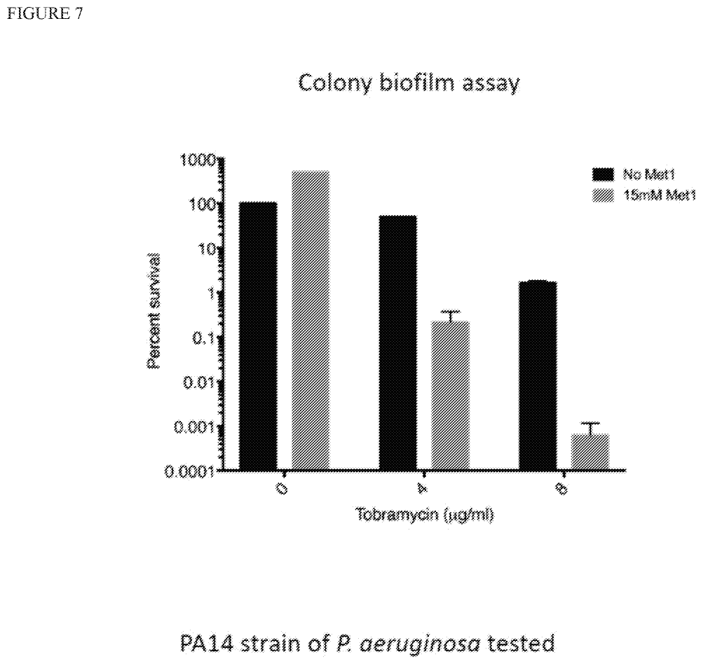

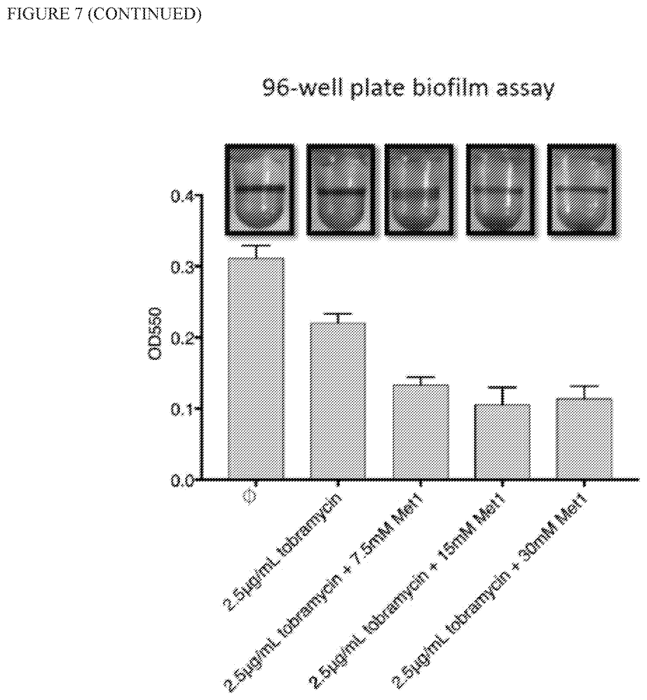

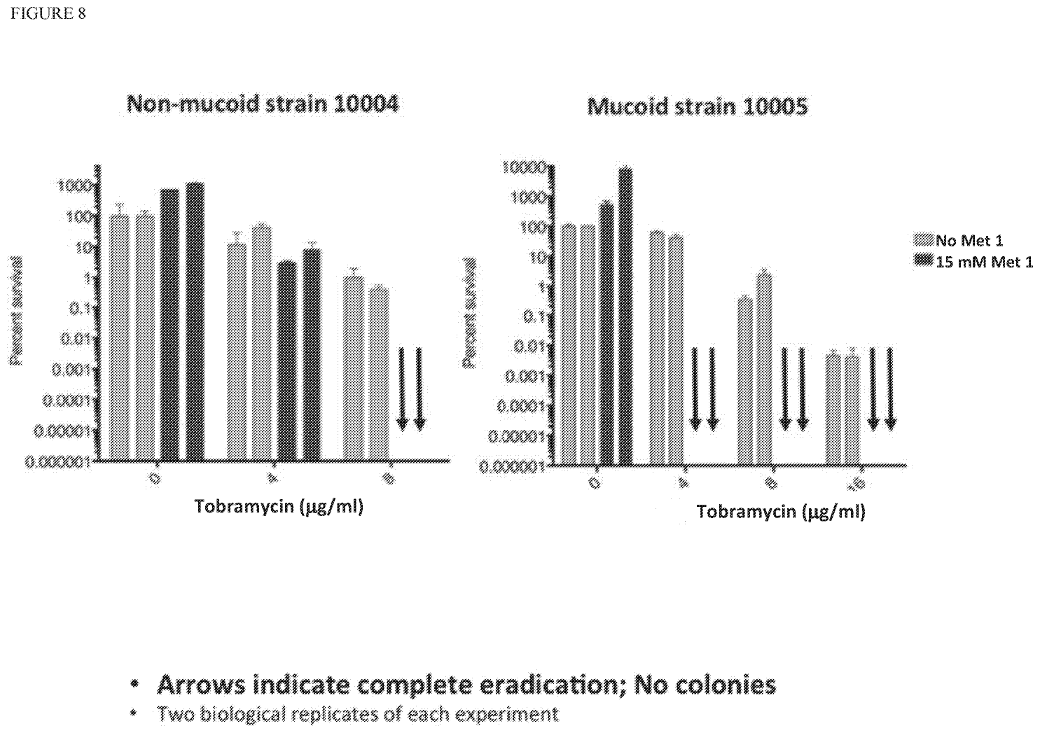

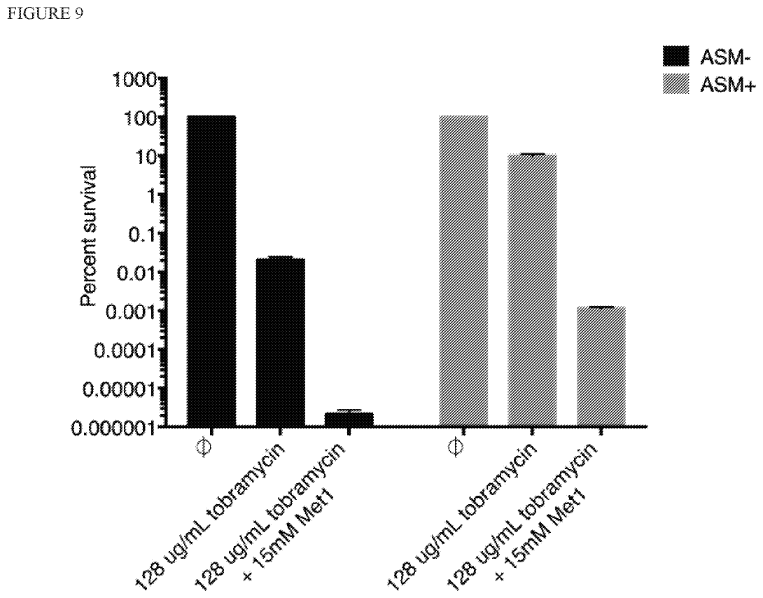

The potentiation effect for tobramycin was tested in two biofilm assays: colony biofilm assay and 96-well plate biofilm assay. As shown in FIG. 7, 15 mM fumarate shows potentiation of tobramycin in both biofilm assays, showing that sufficient fumarate and tobramycin can penetrate bacterial biofilms. Notably, the biofilm assays are naïve cultures in that there is no selection for persisters, although biofilms are expected to be enriched in persisters. Similar results were obtained using colony biofilm assay with both mucoid and non-mucoid CF clinical isolates (FIG. 8), showing eradication of P. aeruginosa in these assays.

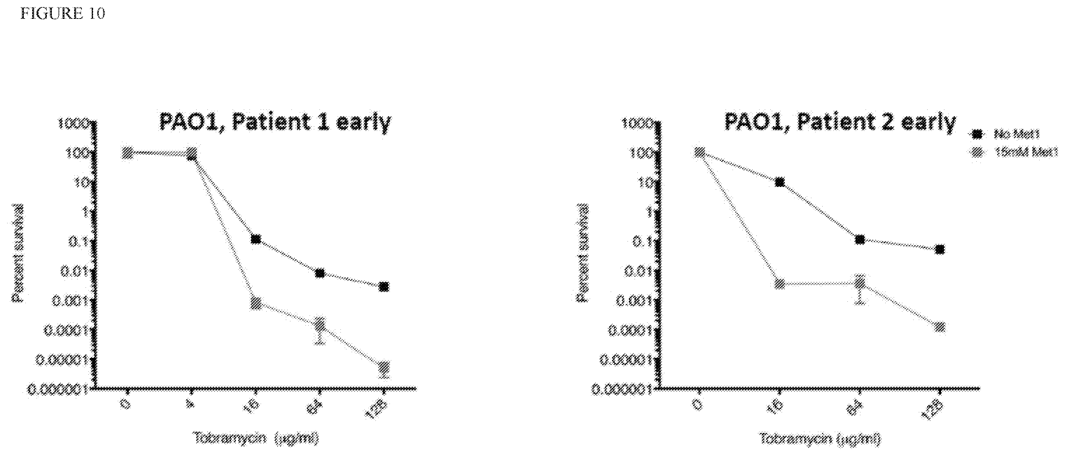

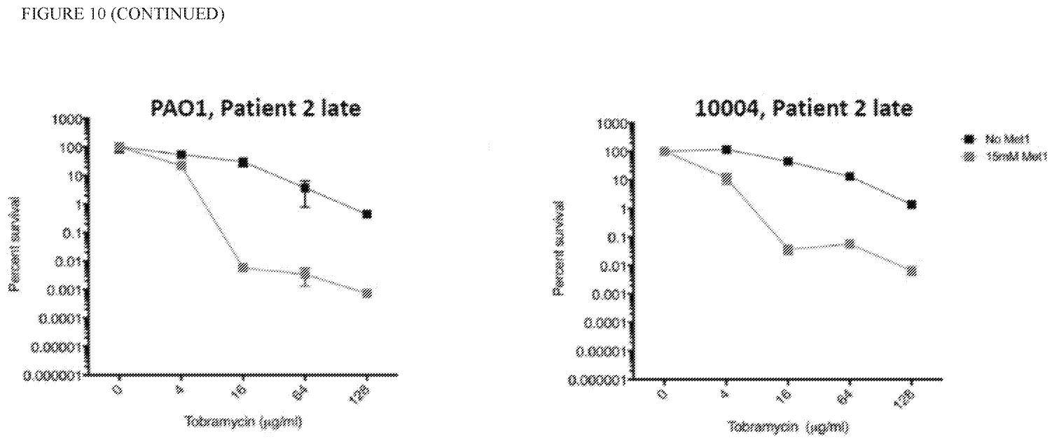

In the presence of artificial sputum media with and without amino acids, some tolerance is observed, but the potentiation of tobramycin with 15 mM fumarate remains an improvement of several logs (FIG. 9). FIG. 10 shows that 3 logs of potentiation is observed in the presence of CF sputum.

1. A method for controlling bacterial infection and/or colonization in the lungs of a patient, the method comprising administering to the lungs of the patient by inhalation of a formulation comprising an aminoglycoside antibiotic selected from tobramycin and amikacin, and a proton-motive force stimulating metabolite; the molar ratio of the aminoglycoside and the metabolite being in the range of from 1:1 to 1:15.

2. The method of claim 1, wherein the formulation is an aqueous solution delivered by a nebulizer.

3. The method of claim 2, wherein the formulation contains tobramycin at from about 100 to about 400 mg per unit dose.

4. The method of claim 3, wherein the formulation contains about 300 mg of tobramycin per unit dose.

5. The method of claim 3, wherein the formulation contains tobramycin at from about 115 to about 250 mg per unit dose.

6. The method of any one of claims 1 to 5, wherein the metabolite is one or a combination of fumarate, pyruvate, methylpyruvate, ethylpyruvate, succinate, glucose, and propionate.

7. The method of any one of claims 3 to 6, wherein the formulation contains from about 100 mg to about 500 mg per dose of the proton motive force stimulating metabolite.

8. The method of claim 7, wherein the metabolite is fumarate optionally in combination with succinate.

9. The method of claim 2, wherein the formulation contains amikacin at from about 200 to about 500 mg per dose.

10. The method of claim 9, wherein the formulation contains about 500 mg of amikacin per dose.

11. The method of claim 9, wherein the formulation contains amikacin at from about 200 to about 350 mg per dose.

12. The method of any one of claims 9 to 11, wherein the metabolite is one or a combination of fumarate, pyruvate, methylpyruvate, ethylpyruvate, succinate, glucose, and propionate.

13. The method of claim 12, wherein the formulation contains from about 100 mg to about 500 mg per dose of the proton motive force stimulating metabolite.

14. The method of claim 13, wherein the metabolite is fumarate optionally in combination with succinate.

15. The method of claim 1, wherein the formulation is a powder.

16. The method of claim 15, wherein the formulation contains tobramycin at from about 75 mg to about 150 mg per dose.

17. The method of claim 15 or 16, wherein the metabolite is one or a combination of fumarate, pyruvate, methylpyruvate, ethylpyruvate, succinate, glucose, and propionate.

18. The method of claim 17, wherein the formulation contains from about 100 mg to about 500 mg per dose of the proton motive force stimulating metabolite.

19. The method of claim 18, wherein the metabolite is fumarate optionally in combination with succinate.

20. The method of claim 15, wherein the formulation contains amikacin at from about 100 to about 200 mg.

21. The method of claim 20, wherein the metabolite is one or a combination of fumarate, pyruvate, methylpyruvate, ethylpyruvate, succinate, glucose, and propionate.

22. The method of claim 21, wherein the formulation contains from about 100 mg to about 500 mg per dose of the proton motive force stimulating metabolite.

23. The method of claim 22, wherein the metabolite is fumarate optionally in combination with succinate.

24. The method of any one of claims 1 to 23, wherein the patient has cystic fibrosis.

25. The method of claim 24, wherein the formulation is administered from one to three times daily.

26. The method of claim 25, wherein the formulation is administered once daily.

27. The method of claim 25, wherein the formulation is administered twice daily.

28. The method of any one of claims 24 to 27, wherein the formulation is delivered for at least 7 consecutive days, and no more than 28 consecutive days.

29. The method of claim 28, wherein the formulation is delivered for 7 to 21 consecutive days.

30. The method of claim 29, wherein the formulation is delivered for about 7 consecutive days, about 14 consecutive days, or for about 21 consecutive days.

31. The method of any one of claims 28 to 30, wherein administration of the formulation is resumed after about 28 days or more.

32. The method of claim 31, wherein administration of the formulation is resumed after about 6 weeks, after about 8 weeks, after about 10 weeks, after about 12 weeks, after about 2 months, after about 3 months, after about 4 months, after about 5 months, or after about 6 months.

33. The method of any one of claims 1 to 23, wherein the patient has non-cystic fibrosis bronchiectasis.

34. The method of claim 33, wherein the formulation is administered from one to three times daily.

35. The method of claim 34, wherein the formulation is administered once daily.

36. The method of claim 34, wherein the formulation is administered twice daily.

37. The method of any one of claims 33 to 36, wherein the formulation is delivered for at least 7 consecutive days, and no more than 28 consecutive days.

38. The method of claim 37, wherein the formulation is delivered for 7 to 21 consecutive days.

39. The method of claim 38, wherein the formulation is delivered for about 7 consecutive days, about 14 consecutive days, or for about 21 consecutive days.

40. The method of any one of claims 1 to 23, wherein the infection comprises a non-tuberculous mycobacterial pulmonary infection.

41. The method of claim 40, wherein the formulation is administered from one to three times daily.

42. The method of claim 41, wherein the formulation is administered once daily.

43. The method of claim 41, wherein the formulation is administered twice daily.

44. The method of any one of claims 40 to 43, wherein the formulation is delivered for at least 7 consecutive days, and no more than 28 consecutive days.

45. The method of claim 44, wherein the formulation is delivered for 7 to 21 consecutive days.

46. The method of claim 45, wherein the formulation is delivered for about 7 consecutive days, about 14 consecutive days, or for about 21 consecutive days.

47. The method of any one of claims 1 to 23, wherein the Pseudomonas infection is associated with chronic obstructive pulmonary disorder (COPD).

48. The method of claim 47, wherein the formulation is administered from one to three times daily.

49. The method of claim 48, wherein the formulation is administered once daily.

50. The method of claim 48, wherein the formulation is administered twice daily.

51. The method of any one of claims 47 to 50, wherein the formulation is delivered for at least 7 consecutive days, and no more than 28 consecutive days.

52. The method of claim 51, wherein the formulation is delivered for from 7 to 21 consecutive days.

53. The method of claim 52, wherein the formulation is delivered for about 7 consecutive days, about 14 consecutive days, or for about 21 consecutive days.

54. The method of any one of claims 1 to 53, wherein the patient is undergoing treatment with an antibiotic that antagonizes aminoglycoside action.

55. The method of claim 54, wherein the patient is undergoing treatment with a macrolide antibiotic.

56. The method of claim 55, wherein the macrolide is azithromycin.

57. A unit dose formulation for delivery by nebulizer, the formulation comprising in an aqueous solution:
from 100 to 400 mg of tobramycin, or from 300 to 600 mg of amikacin; and
from about 100 mg to about 500 mg of one or a combination of metabolites selected from fumarate, pyruvate, methylpyruvate, ethylpyruvate, succinate, glucose, and propionate.

58. The formulation of 57, packaged in ampules of from 2 to 10 ml.

59. The formulation of claim 58, packaged in ampules of from about 2 to about 5 ml.

60. The formulation of any one of claims 57 to 59, comprising a molar ratio of aminoglycoside to metabolite of from 1:1 to 1:15.

61. The formulation of claim 60, comprising from about 115 to about 300 mg of tobramycin, and from about 105 to about 425 mg fumarate in 5 ml aqueous solution.

62. The formulation of claim 61, wherein the formulation contains from 50 to 128 mM tobramycin and from 181 to 727 mM fumarate.

63. A unit dose formulation for delivery by powder aerosol, the formulation is a fine powder comprising:
from 75 to 150 mg of tobramycin, or from 100 to 200 mg of amikacin; and
from about 50 mg to about 250 mg of one or a combination of metabolites selected from fumarate, pyruvate, methylpyruvate, ethylpyruvate, succinate, glucose, and propionate.

64. The unit dose of claim 63, wherein the powder is comprised in capsules.

65. The unit dose of claim 64, wherein 2 to 5 capsules constitute a unit dose.
